(19) United States
(12) Patent Application Publication
(10) Pub. No.: US 2012/0240937 A1
(43) Pub. Date: Sep. 27, 2012

(54) Coating for Latex Articles

(75) Inventors: William Bess, Edsion, NJ (US); Leonard Mackles, New York, NY (US)

(73) Assignee: GlaxoSmithKline LLC, Philadelphia, PA (US)

(21) Appl. No.: 13/513,970

(22) PCT Filed: Dec. 9, 2010
(86) PCT No.: PCT/US10/59625

§ 371 Date: Jun. 5, 2012

Related U.S. Application Data
(60) Provisional application No. 61/285,024, filed on Dec. 9, 2009.

(51) Int. Cl. A61L 31/16; A61L 31/10; C09D 5/14; A61F 6/04

(52) U.S. Cl. 128/844; 106/15.05

(57) ABSTRACT

This invention relates to novel articles, particularly latex barrier articles, being coated with an antimicrobial coating useful to reduce the risk of cross-contamination and/or infection by harmful pathogenic microorganisms.

### FIELD OF THE INVENTION

This invention relates to novel articles, particularly latex barrier articles, being coated with an antimicrobial coating useful to reduce the risk of cross-contamination and/or infection by harmful pathogenic microorganisms. This invention also relates to a process for depositing such coatings, and to compositions for use in such processes.

### BACKGROUND OF THE INVENTION

Pathogenic microbes in humans, including viruses, enter the system of the host predominantly through mucous membranes and the respiratory tract. The first step in any infection is attachment or colonization with subsequent invasion and dissemination of the infectious microbe. Inactivation of potential pathogenic microbes at the site of entry could prevent many infectious diseases.

Barrier articles, such as prophylactic devices, wound dressings and medical gloves all serve a role in preventing pathogenic microbes from penetrating the skin and entering the host. Prophylactic devices, in particular, are widely used for birth control and disease prevention.

Microbicidal and spermicidal compositions have been used in creams, foams and suppositories by themselves or in combination with prophylactic devices primarily for the purpose of contraception. Prophylactic devices for the prevention of sexual disease, as used in large numbers throughout the world, are most often made of natural rubber latex material in very thin wall thicknesses. However, natural rubber material, in such thin wall membranes, does not provide a continuous, impermeable barrier to the passage of micro-size pathogens, such as the virus causing Human immunodeficiency virus (“HIV”) or Herpes simplex virus (“HSV”). Such viruses are known to be as small as 0.1 micromillimeter (0.001 millimeter), or about 250 times smaller than the length of the human sperm and at least 30 times smaller than the thickness of such sperm. The natural rubber membrane is comprised of a polymer matrix characterized by myriads of randomly distributed microsize openings or pores formed among the polymer chains.

Thus, although such natural rubber prophylactics have been found to be an effective barrier preventing the transmission of the larger sperm, there is no assurance that such devices are effective in preventing the transmission of such much smaller viruses, and some testing to date has indicated to the contrary.

Thin polymer films are used in the manufacture of a variety of barrier articles such as condoms, wound dressings, and medical gloves. The application of such a coating to barrier articles should not reduce the ability of such articles to be a primary barrier. The coating should also strongly adhere to the underlying substrate and must be able to be applied to a variety of substrate materials, without degrading the intrinsic properties of the substrate material. In addition, the coating must be able to operate over a physiologically broad spectrum of pH and physiological temperatures.

U.S. Pat. No. 5,700,679 uses a composition containing a lipid vesicle having an outer bilayer formed of a non-ionic amphiphile, a surfactant having anti-viral and/or spermicidal activity, an oil and a sterol, to coat condoms.

U.S. Pat. No. 6,732,735 uses a reticulated coating on various barrier articles to enhance the lubricity with respect to both dry and damp products.

U.S. Pat. No. 6,892,732 relates to a condom coated with a genetically modified or natural lactic acid bacteria-containing composition for disease prevention.

The present inventors have unexpectedly discovered that a coating composition comprising a certain polyol, including propylene glycol and glycerine, or a polymer such as poly(ethylene glycol), a surfactant and an acid, provide antiviral activity in a wide variety of applications, and at least in part provide a solution to the problem of combating viral infection.

### SUMMARY OF THE INVENTION

In a first aspect, this invention is directed to a personal protection barrier article comprising a barrier layer which is substantially impermeable to water and/or air, and which is adapted to provide a physical barrier between a human body and an undesirable contact, the barrier layer having on its surface a coating composition having activity to deactivate pathogenic microorganisms, the coating comprising a polyol, including propylene glycol and/or glycerine, or a polymer such as poly(ethylene glycol), a surfactant and an acid.

In a second aspect, the invention is directed to a process for making such a personal protection barrier article, in which process the surface of the material of the barrier layer is contacted with the composition comprising the polyol, including propylene glycol and/or glycerine, or a polymer such as poly(ethylene glycol), the surfactant and the acid, thereby depositing the coating composition. If the barrier article has multiple layers, including the barrier layer, such a process may be performed either prior to or after incorporating the barrier layer into the barrier article.

In a third aspect, this invention is directed to a coating composition having activity to de-activate pathogenic microorganisms, the coating comprising a polyol, including propylene glycol and/or glycerine, or a polymer such as poly(ethylene glycol), a surfactant and an acid.

In a fourth aspect, this invention is directed to a method for preventing the transfer of a pathogen from one individual to another by wearing a latex barrier article coated with a composition comprising a polyol, including propylene glycol and/or glycerine, or a polymer such as poly(ethylene glycol), a surfactant and an acid.

### DETAILED DESCRIPTION OF THE INVENTION

Examples of materials from which such a barrier layer can be made include soft plastics materials, elastomers such as synthetic and natural rubbers, for example, latex, and fabric, either woven or non-woven, natural or synthetic.

Examples of barrier articles include condoms, wound dressings, human body clothing such as impermeable clothing e.g., for healthcare workers or military personnel and gloves such as rubber gloves.

Examples of fabrics from which such an article of clothing can be made include cotton, rayon, nylon, polyester and fabrics conventionally used for clothing, and such fabrics may be permeable to air, or both air and water, or may be permeable to air but not water. Examples of such clothing include healthcare workers', e.g., physicians', nurses' uniforms and other clothing. Advantageously such clothing may comprise conventional commercially available clothing upon which the coating composition is deposited, and being visually indistinguishable from conventional clothing.

Suitably the fabric may comprise the outer layer of such clothing. The outer layer of clothing is normally the layer with which initial contact of the wearer with a contaminated environment occurs, and providing the fabric as the outer layer can help to neutralize any pathogen which contacts the surface.

It is believed that the coatings of this invention may be effective against the viruses that cause HIV and HSV, and mutated serotypes of these.

As used herein at all occurrences, the term “polyol”, means an alcohol containing multiple hydroxyl groups, including propylene glycol, glycerine (used interchangeably with glycerol), and the like.

The coating and impregnation compositions of this invention include one or more surfactant. A surfactant can facilitate wetting of the articles of the invention, and the contact between the coating composition and the article itself. Pathogens such as virus are known to be carried in small droplets of water, and consequently enhanced wetting of the article can enhance the effective contact between the pathogen and the active materials on the article. Furthermore surfactants are known to be effective in disrupting the membranes of virus and bacteria.

Various types of surfactant may be used in the compositions of this invention. Suitably, a non-ionic surfactant may be used. Examples of non-ionic surfactants are those selected from the Tween™ or Polysorbate™ family (i.e., based on polyoxyethylene sorbitan fatty acid esters such as the monolaurate) of surfactants. Preferred non-ionic surfactants include Polysorbate 20™.

Suitably, an ionic, e.g., anionic surfactant may be used. Such surfactants are compounds having a hydrophilic anionic group and an associated cation. Such a cation may be metallic, such as alkali metal, or non-metallic such as ammonium or quaternary ammonium. Typically such anionic surfactants comprise the hydrophilic anionic group and the cation in the form of a salt. Preferably the anionic surfactant comprises a sodium salt of an organic hydrophilic anionic group. Suitably the organic hydrophilic anionic group may be a sulphonic acid or carboxylic acid group.

A preferred such anionic surfactant compound has the formula (I):

CₙH₂ₙ₊₁—Z⁻M⁺ (I)

where n is 8 to 20, preferably 10 to 15, Z is SO₃ or SO₄, and M is sodium or potassium. A preferred anionic surfactant of this type is sodium lauryl sulphate (n=12, Z is SO₄, M is sodium).

Other anionic surfactants which may be suitable are those of the formula (II):

CₙH₂ₙ₊₁—X—CₘH₂ₘ—Z⁻M⁺ (II)

where n+m are 8 to 20, X is —O— or —CO.O—, Z is SO₃ or SO₄, and M is sodium or potassium. A preferred anionic surfactant of this type is sodium cocoyl isethionate (n=9, m=2, X is CO.O, Z is SO₃, M is sodium. Another anionic surfactant of formula (II) is sodium laureth sulphate.

Other anionic surfactants which may be suitable are those of the formula (III):

CₙH₂ₙ₊₁—CO.NR.—CₘH₂ₘ—Z⁻M⁺ (III)

Where n and m are each 1 or more, n+m are 8 to 20 R is C₁₋₃ alkyl, Z is CO.O, SO₃ or SO₄, and M is sodium or potassium. An example of an anionic surfactants of this type is sodium methyl cocoyl taurate (R is methyl, m=2, Z is SO₃, M is sodium).

Other anionic surfactants are olefin sulphonates such as alpha-olefin such as the commercial material Bioterge™ As-40, being the sodium salt of C₁₄₋₁₆ sulphonates.

Other anionic surfactants which may be suitable include sodium methyl lauroyl taurate, sodium methyl stearoyl taurate and sodium methyl palmitoyl taurate (and their analogues of different alkyl chain length), ammonium lauryl sulphate, ammonium laureth sulphate, sodium cocoyl sarcosinate, triethanolamine lauryl sulphate, triethanolamine laureth sulphate, disodium oleamide sulfosuccinate, disodium laureth sulfosuccinate, disodium dioctyl sulfosuccinate. Other classes of anionic surfactants which may be suitable include the alkaryl sulphonates, alkyl succinates, alkyl sulphosuccinates, N-alkoyl sarcosonates, alkyl phosphates, alkyl ether phosphates, and acyl methyl taurates, especially the sodium, magnesium, ammonium and mono-, di- and tri- ethanolamine salts. Alkyl groups in the preceding may contain 8 to 20 carbon atoms. Alkyl ether sulphates and alkyl ether phosphates may contain 1 to 10 ethylene oxide or propylene oxide units per molecule.

The coating and impregnation compositions of the invention include one or more acid. Suitably the one or more acid may be selected from organic carboxylic acids, preferably a solid such acid. Examples of such solid carboxylic acids include: citric, salicylic, fumaric, benzoic, glutaric, lactic, malonic, acetic, glycolic, malic, adipic, succinic, aspartic, phthalic, tartaric, glutamic, pyroglutamic, gluconic acid, sorbic acid and mixtures of two or more thereof.

It is known to use acids such as citric acid as antiviral agents, and the presence of such an acid can enhance the anti-viral activity of the composition. However it has been found difficult to deposit citric acid on articles because of poor adhesion between the citric acid and the substrate. It has advantageously been found that the polyols , including propylene glycol and glycerine, or a polymer such as poly(ethylene glycol), of the type used in the present invention can act to enhance binding of such acids to substrates.

Suitably, the acid, for example citric acid, is present in a range between 0.25% w/w to 15% w/w. In one embodiment, the range is 1% w/w to 12% w/w. In another embodiment, the range is 5% w/w to 10% w/w.

A preferred combination of polyol, surfactant and acid in the coating and impregnation compositions of this invention is propylene glycol; the non-ionic surfactant Polysorbate 20 (polyoxyethylene sorbitan monolaurate); and the acid citric acid.

The coating and impregnation compositions of this invention may also incorporate one or more antimicrobial compound. Suitable examples of such compounds include sorbic acid, benzoic acid, thymol, antimicrobial oils, quaternary ammonium compounds (e.g., benzalkonium chloride, cetrimide), phenolic compounds (e.g., triclosan, salicylic acid) biguanides (e.g., chlorhexidine, alexidine) and mixtures thereof.

The compositions of this invention are suitably anhydrous, which compositions are not tacky, but provide a desired amount of lubrication and antimicrobial activity.

The ingredients of the coating form a liquid composition, which is applied to the article e.g., by dipping, spraying or other conventional means. The instant composition dries quickly so there is no need for a prolonged drying process, or subsequent drying using heat.

The present invention will now be described by way of example only. Examples of such a liquid coating composition are given below:

### Example 1

Coated condoms were prepared by dipping the first 3-4 inches of the condom into the solution according to Composition 1 and Composition 2, below. The resulting coating averaged about 400 mg, which produced a citric acid content of about 40 mg.

### Composition 1

| Ingredient | % by weight |
|---|---|
| Propylene Glycol | 87.00 |
| Polysorbate 20, EP | 3.00 |
| Citric acid monohydrate, EP | 10.00 |
| Totals: | 100.00 |

### Composition 2

| Ingredient | % (w:w) in solution (as is) |
|---|---|
| Propylene Glycol | 80.00 |
| Polysorbate 20, EP | 10.00 |
| Citric acid monohydrate, EP | 10.00 |
| Totals: | 100.00 |

### Example 2

Coating compositions containing citric acid dissolved in either polyethylene glycol (PEG) or propylene glycol (PG) have been successfully applied to latex condoms. The purpose of the following study was to determine whether these compositions could provide significant (>3 log reduction) antiviral activity against pathogens involved in sexually transmitted diseases, specifically Herpes Simplex Virus-2 (HSV-2) and Human Immunodeficiency virus (HIV-1).

To prepare each of the 3 formulations below, the ingredients were mixed together at 70° C.-75° C. until completely dissolved. The solutions were then diluted 1:10 in ddH₂O prior to testing for antiviral activity.

### Composition 3

| Ingredient | % by weight |
|---|---|
| Polyethylene Glycol 400 (PEG 400) | 80.00 |
| Polysorbate 20 | 10.00 |
| Citric acid monohydrate | 10.00 |
| Totals: | 100.00 |

### Composition 2

| Ingredient | % by weight |
|---|---|
| Propylene Glycol (PG) | 80.00 |
| Polysorbate 20 | 10.00 |
| Citric acid monohydrate | 10.00 |
| Totals: | 100.00 |

### Composition 3

| Ingredient | % by weight |
|---|---|
| Polyethylene Glycol-400 (PEG) | 80.00 |
| Polysorbate 20 | 10.00 |
| Citric acid monohydrate | 10.00 |
| Totals: | 100.00 |

Viruses used were Herpes Simplex Virus type 2 (HSV-2), Strain G, obtained from ATCC (VR-734) and Human Immunodeficiency Virus type 1 (HIV-1), obtained from Advanced Biotechnologies, Inc., Columbia, Md. Suspensions of HSV-2 stock virus were prepared to 4-8 log₁₀ TCID₅₀/mL in Minimum Essential Medium (MEM) supplemented with 5% (v/v) heat-inactivated fetal bovine serum, 10 ug/ml gentamicin, 100 units/ml penicillin, and 2.5 ug/ml amphotericin B. Stock suspensions of HIV-1 were prepared to 4-8 log₁₀ TCID₅₀/mL in RPMI-1640 containing 10% fetal bovine serum).

A suspension of either HIV-1 or HSV-2 was incubated to a 1:10 dilution of each test substance for a 1 minute exposure time. Aliquots were then removed, neutralized by serial dilution, and assayed for the presence of virus by infecting susceptible host cells. Three replicates were performed for each test substance. The positive virus control, cytotoxicity controls, and neutralization controls were assayed in parallel. The Spearman-Karber formula was used to calculate viral loads as TCID₅₀. Antiviral efficacy was calculated from the difference between the geometric mean loads of virus in the solutions versus the original inoculation. The viral loads applied to the compositions in these experiments ranged from 5.5 to 6.25 log₁₀ TCID₅₀ for HSV-2, and 6.25 log₁₀ TCID₅₀ for HIV-1.

For HSV-2 (Table 1), the average viral load of the solutions after a 1 minute exposure time ranged from below the limits of detection (≦1.5 log₁₀ TCID₅₀) to 1.75 log₁₀ TCID₅₀. Therefore, the relative antiviral efficacy ranged from 4.25 to ≧4.50 log₁₀.

For HIV-1 (Table 2), the average viral load in all of the solutions after 1 minute of exposure were below the limits of detection (≦1.5 log₁₀ TCID₅₀) . Therefore, the relative antiviral efficacy was ≧4.75 log₁₀ in all three compositions.

These results confirm the rapid (≦1 minute) and substantial (>4 log reduction) antiviral activity of the three compositions against HIV-1 and HSV-2, which are important sexually transmitted pathogens. The compositions were designed to be applied full strength as a condom lubricant, so the fact that a 1:10 dilution is effective implies that likely lower levers of citric acid (e.g., at approx 1% levels) may also be effective. In addition, both PG (Composition 3; Composition 4) and PEG (Composition 5) containing formulations were effective against both HIV-1 and HSV-2.

TABLE 1
Antiviral Activity Against Herpes Simplex Virus Type 2 in Suspension
Following a One Minute Exposure Period

| | Amt. Virus | | |
|---|---|---|---|
| Full | Inoculated | Amt. Virus Remaining | |
| Thickness | in Solution | after 1 Min Contact Time | Log (%) |
| Condom | (Avg Log | (Log TCID₅₀) | Reduction in |
| Sample | TCID₅₀) | N = 3 | Virus Titer |
| Comp. 3 | 6.0 | (All replicates) ≦1.5, ≦1.5, | ≧4.5 (≧99.997) |
| | | ≦1.5* | |
| Comp. 2 | 6.0 | (All replicates) ≦1.5, ≦1.5, | ≧4.5 (≧99.997) |
| | | ≦1.5* | |
| Comp 3 | 6.0 | (All replicates) 1.75, 1.75, | 4.25 (99.994) |
| | | 1.75 | |

*Limit of detection = 1.5 log Log TCID₅₀

TABLE 2
Antiviral Activity Against Human Immunodeficiency Virus-1 in
Suspension Following a One Minute Exposure Period

| | Amt. Virus | | |
|---|---|---|---|
| Full | Inoculated | Amt. Virus Remaining | |
| Thickness | in Solution | after 1 Min Contact Time | Log (%) |
| Condom | (Avg Log | (Log TCID₅₀) | Reduction in |
| Sample | TCID₅₀) | N = 3 | Virus Titer |
| Comp. 3 | 6.25 | (All replicates) ≦1.5, ≦1.5, | ≧4.75 (≧99.998) |
| | | ≦1.5* | |
| Comp. 2 | 6.25 | (All replicates) ≦1.5, ≦1.5, | ≧4.75 (≧99.998) |
| | | ≦1.5* | |
| Comp 3 | 6.25 | (All replicates) ≦1.5, ≦1.5, | ≧4.75 (≧99.998) |
| | | ≦1.5* | |

*Limit of detection = 1.5 log Log TCID₅₀

It should be understood that the foregoing description is only illustrative of the present invention. Various alternatives and modifications can be devised by those skilled in the art without departing from the present invention. Accordingly, the present invention is intended to embrace all such alternatives, modifications and variations that fall within the scope of the appended claims.

1. A personal protection latex barrier article having on its surface a coating composition for deactivating pathogenic microorganisms, the coating comprising a carrier which is a polyol selected from propylene glycol and glycerine, or a polymer such as poly(ethylene glycol), a surfactant and an acid.

2. The personal protection latex barrier according to claim 1 being a condom.

3. The personal protection latex barrier according to claim 2, wherein the pathogenic microorganism which is deactivated is selected from a herpes virus and human immunodeficiency virus.

4. The personal protection latex barrier according to claim 1, wherein the polyol is propylene glycol.

5. The personal protection latex barrier according to claim 1, wherein the acid is selected from organic carboxylic acids.

6. The personal protection latex barrier according to claim 5, wherein the acid is citric acid.

7. The personal protection latex barrier according to claim 1, wherein the surfactant is a non-ionic surfactant.

8. The personal protection latex barrier according to claim 7, wherein the surfactant is Polysorbate 20.

9. A composition comprising a carrier which is a polyol selected from propylene glycol and glycerine, or a polymer such as poly(ethylene glycol), a surfactant and an acid.

10. A method for preventing the transfer of a pathogen from one individual to another by wearing a latex barrier article coated with a composition comprising a polyol, including propylene glycol and glycerine, or a polymer such as poly(ethylene glycol), a surfactant and an acid.
